(19) United States
(12) Patent Application Publication
(10) Pub. No.: US 2024/0342326 A1
(43) Pub. Date: Oct. 17, 2024

(54) NASAL SWAB APPARATUS AND METHOD FOR APPLYING A CLEANING SOLUTION

(71) Applicant: John W. Polley, Maple City, MI (US)

(72) Inventor: John W. Polley, Maple City, MI (US)

(21) Appl. No.: 18/751,944

(22) Filed: Jun. 24, 2024

Related U.S. Application Data
(63) Continuation-in-part of application No. 18/605,418, filed on Mar. 14, 2024.
Application No. 18/605,418 is a continuation of application No. PCT/US2023/062895, filed on Feb. 20, 2023.
(60) Provisional application No. 63/268,315, filed on Feb. 22, 2022.

(51) Int. Cl. A61L 2/00; A61B 17/24; A61F 13/38; A61K 8/02; A61K 8/43; A61K 8/49; A61L 2/26; A61L 101/34; A61L 101/44; A61Q 17/00

(52) U.S. Cl. CPC A61L 2/0088; A61B 17/24; A61F 13/38; A61K 8/0208; A61K 8/43; A61K 8/498; A61L 2/26; A61Q 17/005; A61B 2017/246; A61L 2101/34; A61L 2101/44; A61L 2202/16; A61L 2202/17; A61L 2202/181

(57) ABSTRACT

A nasal swab kit for cleaning and sanitizing human nose nasal vestibules includes a pair of nasal swab devices, each of the devices having a body from which two spaced apart prongs extend, and each of the prongs having a free end to which a swab is attached. The swabs of a first of the devices are saturated with at least one cleaning ingredient selected from sterile water, glycerin, sodium chloride, sodium benzoate, PEG 60 hydrogenated castor oil, cocamidopropyl betaine and lavender oil. The swabs of a second of the devices are saturated with a sanitizing ingredient such as isopropyl alcohol or mupirocin. The kit includes a first packet inside which the first device is sealed and a second packet inside which the second device is sealed wherein the packets are conjoined at a perforated seam.

### CROSS-REFERENCE TO RELATED APPLICATIONS

This application is a continuation-in-part of the co-pending U.S. patent application Ser. No. 18/605,418 filed Mar. 14, 2024, that is a continuation of the co-pending international patent application Serial No. PCT/US2023/062895 filed Feb. 20, 2023. This application claims the benefit of U.S. provisional patent application Ser. No. 63/268,315 filed Feb. 22, 2022.

Incorporated by reference are U.S. provisional patent application Ser. No. 63/268,315 filed Feb. 22, 2022, PCT international application Serial No. PCT/US2023/062895 filed Feb. 20, 2023, U.S. design patent application Ser. No. 29/909,427 filed Aug. 4, 2023, and U.S. patent application Ser. No. 18/605,418 filed Mar. 14, 2024.

### FIELD OF THE INVENTION

This invention relates to nasal swabs and methods of using nasal swabs. More particularly, the invention is directed to a double prong nasal swabs with a columellar (Nasal, Alar, Nasal Rim, Nasal Opening, Nasal Vestibular, Vestibular Rim) guard and a nasal cleaning solution and a method for the cleaning, washing, prepping, decolonizing, and/or sterilizing of the human nose and nasal vestibules.

### BACKGROUND OF THE INVENTION

This section provides background information related to the present disclosure which is not necessarily prior art.

A Hospital Acquired Infection (HAI) is the dreaded result of a patient who did not have an infection upon admission to a hospital but developed an infection during their hospital stay. HAIs are linked with potential high morbidity and mortality. It is estimated that 1 in 31 hospital patients develop an HAI, affecting 2 million patients yearly and accounting for over 100,000 deaths in the United States alone. The annual financial burden for treating HAIs is estimated at over $40 billion annually (see Publications 1-3 listed below). Hospitals in particular feel this financial burden as the expenses for treating HAIs are often not reimbursable through the patient's insurance and in fact the Center for Medicare Services (CMS) penalizes those hospitals with the highest HAI rates (see Publication 1).

Based in part on the financial incentive to reduce HAIs, hospitals over the past two decades have changed from the concept of most HAIs being an unpreventable “cost of business” to the understanding that the majority of HAIs are preventable. The focus for hospital programs has now shifted from infection control to infection prevention. HAI data is now followed closely by the Center for Disease Control (CDC) through national and state HAI progress reports from individual hospitals (see Publications 1, 2).

HAIs are separated into categories including post-operative surgical site infections (SSI), respiratory/pulmonary infections, access line infections including peripheral and central lines, and catheter related infections most commonly of the genitourinary tract. It is well understood that with postoperative surgical site infections the source of the infectious agent or bacteria often arises from an endogenous source from the patient themselves.

Gram-positive cocci including Staphylococcus aureus and Methicillin resistant Staphylococcus aureus (MRSA) are the most common organisms initially involved in postop surgical site infections (see Publication 4). Gram-positive cocci like all bacteria thrive in and an environment that is warm, has moisture or water, and somewhere that avoids light exposure. In addition, like all living organisms, bacteria need nutrition for sustainability and propagation.

The ideal locations in the human body that supply all of these requirements are the hair bearing skin areas of the groin, axillae, and nasal vestibules. These three areas are protected from the outside environment, are warm, moist, dark, and are abundant in apocrine glands and eccrine glands that can supply the basic nutrient requirements for bacteria along with lipids and proteins from the desquamating epithelium of the skin.

Preoperative body washes focus on thorough mechanical and disinfectant cleaning of the axilla and groin as does an appropriate preoperative surgical prep technique. Cross-contamination from these areas of the body to other sites of HAIs likely plays a very important role in the initiation of these infections as well.

In 1846 Ignaz Semmelweis linked the importance of hand hygiene to infection prevention. Recently most evaluations on strategies to prevent HAIs have focused on handwashing and its critical importance in the prevention and spread of infectious diseases in the hospital and public setting. It is important to realize that infectious organisms on the hand, in and of themselves, do not cause infections. They become infectious only when they are physically transferred from the hand to an open body source such as a fresh incision or open wound, a skin piercing line, a catheter conduit, or access to enter the pulmonary respiratory system.

The other major hair bearing area of the body that houses infectious viruses and bacteria is the nose. Today we are beginning to understand that nasal hygiene is as, or perhaps more, important than hand hygiene for good health and for the prevention of respiratory and other HAIs. 90% of the air we breathe in is through our nose. This air first enters the anterior nose or nasal vestibule which are lined with skin containing apocrine glands, eccrine glands, hair and hair follicles. These hairs and sticky secretions in the nasal vestibules form our body's natural air filter, trapping contagious viruses, bacteria, and allergens that we breathe in or transfer in from hand to face contact.

It has been shown that the nasal vestibules, when clean, are very efficient at trapping particles 0.5 μm or greater (see Publications 5-7). This includes all allergy danders, and bacteria. A lone virus itself has a diameter less than 0.5 μm but typically viruses are transported by an aerosol with particles that range from 0.5 μm or greater (see Publication 7). It is well-documented that the anterior nose is much more heavily colonized by bacteria and any other site in the upper respiratory tract (see Publication 8). Germs can incubate in the nasal vestibules (incubation periods typically 2-6 days) and then can become infectious spreading to the host or transmitted to others (see Publication 8). As with hand washing daily nasal hygiene for suppression of these germs early in the incubation process prior to becoming infectious is the goal. But what is an effective, safe, and easily performed technique for cleaning the nose that doesn't require constant medical supervision.

Nasal disinfection or decolonization is now recognized as an essential component of prophylactic hygiene for pre-operative patients, hospitalized patients, and the general public following exposure to potential sources of airborne viruses and bacteria (crowds, airplanes, close public contact, known carriers, etc.). The main disinfectants utilized for nasal decolonization in the medical industry are alcohol, Povidone Iodine, and a topical antibiotic, mupirocin. Mupirocin application for 5 days peri-operatively has been shown to be most effective in decolonizing the nose of Staphylococcus aureus and MRSA (see Publications 4, 9, 10).

Methodology: All publications on nasal decolonization have focused on the decolonizing agent and not on the technique or method of decolonization. This is a crucial component. All current nasal decolonization techniques simply apply their decolonizing agent onto a dirty nasal vestibular surface. The sanitizing agents (alcohol, Povidone Iodine, mupirocin), when placed or simply painted onto unclean nasal vestibular skin can be largely absorbed by the nasal carriage, significantly decreasing their sanitizing efficiency. The benefit of the mechanical cleaning step is well demonstrated in the article by Rezapoor who in a randomized prospective study showed that cleaning the nasal vestibule with only a saline swab resulted in a negative culture for Staphylococcus aureus in 31% of patients at 4 hours after treatment (see Publication 11).

The medical and nonprofessional literature lacks studies on how to properly decolonize or sanitize the nose. The reason is that published authors in this field have no experience on how to prep/sterilize/clean the nasal vestibules. What is needed is a simple, effective, well documented true surgically proven nasal cleaning and disinfecting technique with the appropriate utensil that can eradicate harmful bacteria, viruses, and allergic dander's in the nasal vestibule during the early colonization phase, prior to overt infection.

### BIBLIOGRAPHY OF PUBLICATIONS

1. Center for Disease Control: Current HAI Progress Report. 2021 www.cdc.gov
2. Stone P W et. al. CMS changes in reimbursement for HAI's. Med Care, 2010 May: 48 (5); 433-439
3. Cohen C C, et. al. Financial incentives to reduce hospital-acquired infections under alternative payment arrangements. Infect Control Hosp Epidemiol, 2018 May; 39 (5): 509-515
4. Septimus E J. Nasal decolonization: What antimicrobials are most effective prior to surgery? Am J. of Infection Control 47 (2019) A53-A57
5. Hatch T F, Gross P. Pulmonary deposition and retention of inhaled aerosols. NY, London Acedemic press. 1964
6. Xie X, et al. How far droplets can move in indoor environments-revisiting the Wells evaporation-falling curve. Indoor Air 2007; 17:211-225. DOI: 10.1111
7. Zhou Y, et al. Particle size distribution and inhalation dose of liquids under selected operating conditions. Inhal Toxicol, 2007; 19:333-342. DOI: 10.1080
8. Wertheim H F, et al. The role of nasal carriage in Staph. Aureus infections. Lancet Infect Dis 2005:5:751-62
9. Gussin G M, et. al. Reducing hospitalizations and multi-drug resistant organisms via reginal decolonization in hospitals and nursing homes. JAMA Online publication. E 1-14. Apr. 1, 2024
10. Septimus E J, Schweizer, ML. Decolonization in prevention of health care-associated infections. Clinical Microbiology Reviews. April 2016, Vol 29 #2 pp201-221.
11. Rezapoor M, Nicholson T, Tabatabaee R M, Chen A F, Maltenfort M G, Parvizi J; Povidone-Iodine-Based Solutions for Decolonization of Nasal Staphylococcus aureus: A randomized, Prospective, Placebo-Controlled Study; J. Arthroplasty, 2017 Sep.; 32 (9): 2815-2819 PMID: 28578841

### BRIEF SUMMARY OF THE INVENTION

According an embodiment of the present invention, a “double prong/dual swab” nasal swab includes a columellar (Nasal, Alar, Nasal Rim, Nasal Opening, Nasal Vestibular, Vestibular Rim) guard or stop, and an antiseptic or disinfectant cleaning solution.

A complete nasal surgical prep/nasal decolonization/nasal hygiene system, with instructions on use, is the goal of the nasal swab apparatus according to the invention. It offers the following advantages:
Professional (MD) surgically based nasal cleaning and decolonization method
Easy to use, foolproof
Compact, lightweight, disposable, inexpensive, safe
Can be used anytime, anywhere
No need for sinks, water, private space to use
Assures proper placement of the device only in the nasal vestibule

A device according to the invention is a double prong swab which consists of consists of a handle and two prongs with a swab on each end of the prongs. The handle is for holding the device with human fingers or hands. The superior border of the handle between the two prongs acts as a nasal or columellar guard to prevent entry of the prongs too far into the nose. The columellar guard of the handle assures proper placement of the swabs, at the end of the prongs, only in the nasal vestibular skin and not higher into the nasal mucosa, or too shallow of placement in the nasal vestibules. The swab is for intranasal or nasal vestibular use or placement. Accurate anatomic placement of the swab(s) ensures thorough cleaning, scrubbing, prepping, disinfecting, and/or decolonizing of the entire nasal vestibule(s).

The device can be constructed from any type of material such as paper or paper product, plastic of any type, metal, synthetic product or paper. The device can be made of biodegradable or non-biodegradable materials. The device has attached to or as part of it a swab, pad, wipe, gauze, wad, foam, sponge, cushion, mop, bolster, and/or covering.

The device is an evidence based daily use nasal vestibular hygiene decolonization apparatus for performing a method and technique that safely and effectively cleans the nose the correct way. The method is a 2-step procedure which adheres to the surgically documented superiority of a 2-step, dual prep technique, for skin sanitation and decolonization. The device consists of two pre-saturated dual swab devices, with each double-pronged device packaged in a separate packet. Two packets, step 1 and step 2, each containing a separate presaturated dual swab device. The packets are conjoined as a single two piece packet with a perforated seam between the two packets which allows for easy separation of step 1 and step 2 packets if desired. Alternatively each packet or Step can come as separate entities. Step 1 is the cleaning step with the dual swabs saturated with the ingredients: sterile water; glycerin; sodium chloride; sodium benzoate; PEG (polyethylene glycol) 60 hydrogenated castor oil; cocamidopropyl betaine; and lavender oil. Alternatively Step 1 may be saturated with alcohol (62% (+/−) ethyl or 70% (+/−) isopropyl) and sterile water. Step 2 is the sanitizing step with the ingredients: mupirocin (1-10% (+/−), a carrier agent (PEG of varying %) and sterile water. Alternatively Step 2 may be saturated with a various percentage of chlorhexidine and an associated carrying agent and sterile water. The two step nasal decolonization/hygiene technique includes: Step 1 cleaning the nasal vestibular skin of carriage with the step 1 device; and Step 2 sanitizing a now clean nasal vestibular lining with the step 2 device. This method/technique avoids the cross and recontamination of other techniques and, for the first time, offers an evidence based surgical protocol for nasal decolonization. The unique dual swab design of the devices has a columellar guard, assuring proper placement only in the nasal vestibular skin, not higher up into the more fragile nasal mucosa. The device is portable, disposable, can be used anytime, anywhere discreetly without the need running water, or other utensils or vesicle containing solutions.

The device can be packaged in, held in, delivered in, wrapped in, parceled in, a plastic or paper or metallic (or other synthetic based material) container with rip off or seal access. The package can contain antiseptic(s) or antibiotics that soak the swab(s) of the device in a various fashions. The package or kit can contain one, two, or more of the devices.

The device can be used to perform a method for the cleaning, washing, prepping, decolonizing and/or sterilizing of the human nose regardless of the sex or age of the person. The method can be used in medicine as a surgical prep/sterilization technique. The method can utilize alone or in any combination, FDA approved surgical prep antiseptics or disinfectants or antibiotics and/or moisturizing agents.

The nasal columellar guide is formed by the superior border of the handle between the two prongs.

The body and/or the prongs are constructed from a material selected from a paper, a paper product, a plastic, a metal and a synthetic product. The material can be biodegradable.

The swab is adapted to hold a quantity of an antiseptic or a disinfectant and to release the antiseptic or the disinfectant upon contact with a surface in the nasal vestibule of the human nose.

According to a first aspect of the invention, a two-step method for cleaning and sanitizing two nasal vestibules of a human nose comprises the steps of: simultaneously cleaning a nasal vestibular lining of carriage in the two nasal vestibules using a first nasal swab device saturated with at least one cleaning ingredient; and simultaneously sanitizing the cleaned nasal vestibular linings using a second nasal swab device saturated with a cleaning ingredient. Each of the cleaning step and the sanitizing step is performed by inserting swabs of the respective first and second steps into the two nasal vestibules, placing the swabs against the vestibular skin linings, rotating the swabs in in a circular motion by moving along a circular horizontal path without rotating about a longitudinal axis of the respective device. The nasal vestibular skin is scrubbed in this fashion with each double swab, scrubbing or washing the nasal vestibular skin for approximately 30 (+/−) seconds for each double swab step.

According to a second aspect of the invention, the first nasal swab device has a pair of swabs saturated with sterile water, glycerin, sodium chloride, sodium benzoate, PEG 60 hydrogenated castor oil, cocamidopropyl betaine and lavender oil. Alternatively Step 1 may be saturated with alcohol (62% (+/−) ethyl or 70% (+/−) isopropyl) and sterile water. Step 2 is the sanitizing step with the ingredients: mupirocin (1-10% (+/−), a carrier agent (PEG of varying %) and sterile water. Alternatively Step 2 may be saturated with a various percentage of chlorhexidine and an associated carrying agent and sterile water.

According to a third aspect of the invention, a nasal swab kit for cleaning and sanitizing human nose nasal vestibules comprises: a pair of nasal swab devices, each of the devices having a body from which two spaced apart prongs extend, and each of the prongs having a free end to which a swab is attached; wherein the swabs of a first of the devices are saturated with at least one cleaning ingredient and the swabs of a second of the devices are saturated with a sanitizing ingredient; a first packet inside which the first device is sealed and a second packet inside which the second device is sealed; wherein the first packet and the second packet are conjoined at a perforated seam; and wherein the first packet and the second packet each have a notch formed in an edge thereof providing starting points for tearing open the packets.

According to a fourth aspect of the invention, the at least one cleaning ingredient is selected from sterile water, glycerin, sodium chloride, sodium benzoate, PEG 60 hydrogenated castor oil, cocamidopropyl betaine and lavender oil or alternatively may be saturated with alcohol (62% (+/−) ethyl or 70% (+/−) isopropyl) and sterile water. The sanitizing step with the ingredients: mupirocin (1-10% (+/−), a carrier agent (PEG of varying %) and sterile water or alternatively may be saturated with a various percentage of chlorhexidine and an associated carrying agent and sterile water.

According to a fifth aspect of the invention, the swabs of the first device are saturated with sterile water, glycerin, sodium chloride, sodium benzoate, PEG 60 hydrogenated castor oil, cocamidopropyl betaine and lavender oil. Alternatively the first device may be saturated with alcohol (62% (+/−) ethyl or 70% (+/−) isopropyl) and sterile water.

According to a sixth aspect of the invention, the swabs of the second device are saturated with the ingredients: mupirocin (1-10% (+/−), a carrier agent (PEG of varying %) and sterile water or alternatively may be saturated with a varying percentage of chlorhexidine and an associated carrying agent and sterile water.

According to a seventh aspect of the invention, the first packet is identified by a “step 1 clean” label on an outside surface thereof and the second packet is identified by a “step 2 sanitize” label on an outside surface thereof.

### BRIEF DESCRIPTION OF THE SEVERAL VIEWS OF THE DRAWING

The above as well as other advantages of the invention will become readily apparent to those skilled in the art from the following detailed description of a preferred embodiment when considered in the light of the accompanying drawings in which:

FIG. 1 is a front view of a nasal swab device according to the invention;

FIG. 2 is a front view of alternate embodiments of the nasal swab device shown in FIG. 1;

FIG. 3 is a schematic view a nasal swab kit including two nasal swab devices according to the invention; and

FIG. 4 is a flow diagram of a method for using the nasal swab kit of FIG. 3 according to the invention.

### DETAILED DESCRIPTION OF THE INVENTION

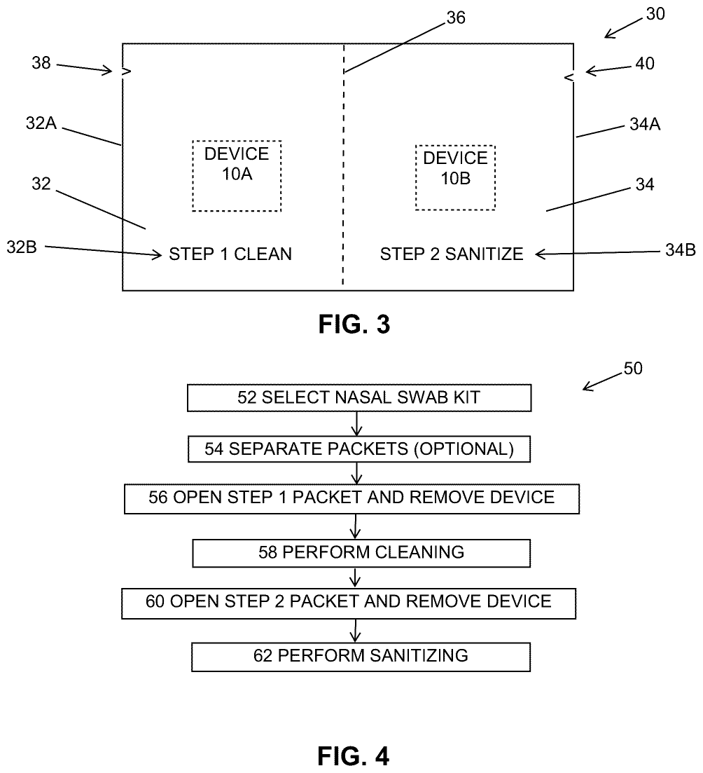

Various alternative embodiments of the invention are disclosed in detail in the U.S. provisional patent application Ser. No. 63/268,315 filed Feb. 22, 2022 and the international patent application Serial No. PCT/US2023/062895 filed Feb. 20, 2023, both incorporated by reference. For example, see FIGS. 3-10 of the international application.

The above as well as other advantages of the present invention will become readily apparent to those skilled in the art from the following detailed description of a preferred embodiment when considered in the light of the accompanying drawings. The following detailed description and included drawings describe and illustrate various exemplary embodiments of the invention. The description and drawings serve to enable one skilled in the art to make and use the invention, and are not intended to limit the scope of the invention in any manner. In respect of the methods disclosed, the steps presented are exemplary in nature, and thus, the order of the steps is not necessary or critical.

More particularly, the invention is directed to single and multiple prong nasal swab devices with a columellar guard, a nasal cleaning solution, and a method for the cleaning, washing, prepping and/or sterilizing of the human nose nasal vestibules.

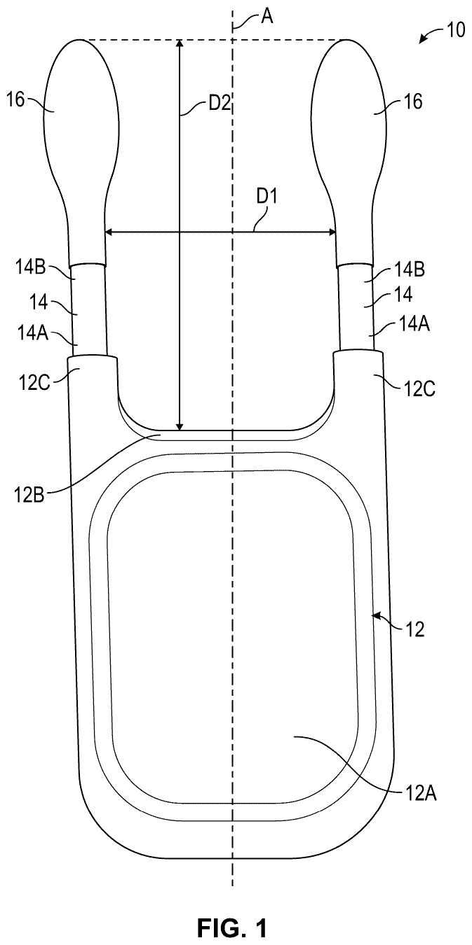

Shown in FIG. 1 is front side of a first embodiment of the nasal swab device 10 according to the invention. The rear side (not shown) can be identical. An elongated body 12 has a longitudinal axis A and is shaped as a handle or stem to be grasped between a thumb and a forefinger of a human hand (not shown). The body 12 can have central depressions 12A formed on opposite sides thereof as gripping aids. An attachment edge 12B of the body 12 has a pair of prongs or arms 14 extending therefrom generally parallel to the longitudinal axis A. The prongs 14 are spaced apart at a predetermined distance D1 to permit insertion into the nostrils of a human nose. Each of the prongs 14 has an attachment end 14A connected to the body 12 at the attachment edge 12B. The prongs 14 can be formed integral with the body 12. In the alternative, the prongs 14 can be separate parts with the attachment ends 14A either attached directly to the edge 12B or inserted into receptacles 12C extending from the edge 12B as shown in FIG. 1. When inserted in the receptacles 12C, the prongs 14 can be fixed or can be removable for replacement. Although the prongs 14 are shown as being of approximately the same length, they could be formed with different lengths.

Each of the prongs 14 has a free end 14B opposite the attachment end 14A. Each of the free ends 14B has an associated swab 16 attached thereto. Thus, the nasal swab device or apparatus 10 can be engaged by a human hand at the body 12 to position the swabs 16 simultaneously into separate ones of the nasal vestibules of a human nose. The portion of the attachment edge 12B that extends transversely between the attachment ends 14A functions as a nasal columellar guide, guard or stop. The nasal columellar guide limits an insertion depth D2 of the prongs 14 and swabs 16 into the nasal vestibules.

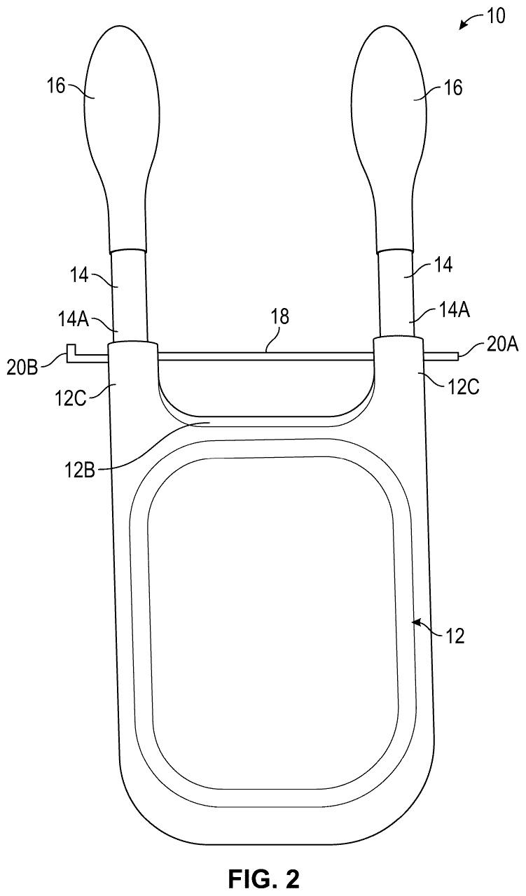

FIG. 2 shows two variations or alternate embodiments of the two prong nasal swab device 10 shown in FIG. 1. In a first variation, instead of the portion of the attachment edge 12B that extends transversely between the attachment ends 14A functioning as the nasal columellar guide, guard or stop, a separate nasal columellar guide 18 is spaced from the attachment edge 12B and extends between the receptacles 12C. In a second variation, a nasal alar guide, guard, stop is provided at each of the receptacles 12C. A nasal alar guide 20A extends from the right side receptacle 12C transverse to a longitudinal axis of the adjacent prong 14. A nasal alar guide 20B extends from the right side receptacle 12C transverse to a longitudinal axis of the adjacent prong 14. The guide 20B has an L-shape or could be curved. The nasal swab device 10 can be provided with any one or more of the guides 18, 20A and 20B.

The device 10 is used as a nasal vestibular hygiene decolonization apparatus for performing a method and technique that safely and effectively cleans the nose the correct way. The method is a 2-step procedure which adheres to the surgically documented superiority of a 2-step, dual prep technique, for skin sanitation and decolonization. The method can be performed using two pre-saturated double prong devices 10. Step 1 is the cleaning step with a first double prong device 10 having the two swabs 16 saturated with the cleaning ingredients: sterile water; glycerin; sodium chloride; sodium benzoate; PEG 60 hydrogenated castor oil; cocamidopropyl betaine; and lavender oil or alternatively may be saturated with alcohol (62% (+/−) ethyl or 70% (+/−) isopropyl) and sterile water. Step 2 is the sanitizing step with a second double prong device 10 having the two swabs 16 saturated with the sanitizing ingredients: mupirocin 1-10% (+/−), a carrier agent (PEG of varying %) and sterile water or alternatively may be saturated with a various percentage of chlorhexidine and an associated carrying agent and sterile water.

The 2-step nasal decolonization/hygiene technique includes: Step 1 cleaning the nasal vestibular skin of carriage with the step 1 device 10; and Step 2 sanitizing a now clean nasal vestibular lining with the step 2 device 10. This method/technique avoids the cross contamination and recontamination of other techniques and, for the first time, offers an evidence based surgical protocol for nasal decolonization. The unique dual swab design of the device 10 has a columellar guard 12B, 18, assuring proper placement only in the nasal vestibular skin, not higher up into the more fragile nasal mucosa. The device 10 is portable, disposable, can be used anytime, anywhere discreetly without the need of running water, or other utensils or vesicle containing solutions.

The step 1 device and the step 2 device can be provided as presaturated double prong devices 10 each in a separate sealed packet. As shown in FIG. 3, a nasal swab kit 30 includes a step 1 packet 32 releasably connected to a step 2 packet 34 along a perforated seam or tear line 36 at adjacent vertical edges of the packets. The perforated seam 36 allows for easy separation of the step 1 packet 32 from the step 2 packet 34 if desired. Sealed in the step 1 packet 32 is a step 1 device 10A having each swab presaturated with the cleaning ingredients described above. A notch 38 is formed in a packet edge 32A opposite the perforated seam 36 as a starting point for tearing open the packet 32. Sealed in the step 2 packet 34 is a step 2 device 10B having each swab presaturated with the sanitizing ingredients described above. A notch 40 is formed in a packet edge 34A opposite the perforated seam 36 as a starting point for tearing open the packet 34. The step 1 packet 32 is identified by a “STEP 1 CLEAN” label 32B on the outer surface. The step 2 packet 34 is identified by a “STEP 2 SANITIZE” label 34B on the outer surface.

The method steps for performing the 2-step nasal decolonization/hygiene technique according to the invention are shown in FIG. 4. The method 50 includes a first step 52 of selecting the nasal swab kit 30. In an optional second step 54, the conjoined packets 32, 34 can be separated at the perforated seam 36 if desired. In a third step 56, the step 1 packet 32 is opened by tearing horizontally starting at the notch 38 and the device 10A is removed from the packet. In a fourth step 58 the simultaneous cleaning of the nasal vestibular skin of carriage with the step 1 device 10A is performed. A preferred technique for using the devices 10A, 10B is to hold the handle 12 between the thumb and index finger of your hand. Then insert both swabs 16, one into each nostril, placing them against the nostril skin. The device 10A is then rotated in a circular motion by moving along a circular horizontal path without rotating about a longitudinal axis of the respective device. The nasal vestibular skin is scrubbed in this fashion, mechanically scrubbing or washing the nasal vestibular skin for approximately 30 (+/−) seconds. Now the device 10A can be discarded.

In a fifth step 60, the step 2 packet 34 is opened by tearing horizontally starting at the notch 40 and the device 10B is removed from the packet. In a sixth step 62 the simultaneous sanitizing of a now clean nasal vestibular lining with the step 2 device 10B is performed. The handle 12 is held as described above. Then both swabs 16 are inserted, one into each nostril, placing them against the nostril skin. The device 10B is then rotated in a circular motion by moving along a circular horizontal path without rotating about a longitudinal axis of the respective device. The nasal vestibular skin is scrubbed in this fashion, mechanically scrubbing or washing the nasal vestibular skin for approximately 30 (+/−) seconds. Now the device 10B can be discarded.

The cleaning step involves using the double prong device 10, 10A with the dual swabs 16 saturated with the ingredients: sterile water; glycerin; sodium chloride; sodium benzoate; PEG 60 hydrogenated castor oil; cocamidopropyl betaine; and lavender oil or alternatively may be saturated with alcohol (62% (+/−) ethyl or 70% (+/−) isopropyl) and sterile water. The sanitizing step 2 involves using the double prong device 10, 10B with the dual swabs 16 with the ingredients: mupirocin 1-10% (+/−), a carrier agent (PEG of varying %) and sterile water or alternatively may be saturated with a various percentage of chlorhexidine and an associated carrying agent and sterile water. The 2-step nasal decolonization/hygiene technique includes: Step 1 cleaning the nasal vestibular skin of carriage with the step 1 device; and Step 2 sanitizing a now clean nasal vestibular lining with the step 2 device. This method/technique avoids the cross and recontamination of other techniques and, for the first time, offers an evidence based surgical protocol for nasal decolonization. The unique dual swab design of the devices has a columellar guard, assuring proper placement only in the nasal vestibular skin, not higher up into the more fragile nasal mucosa. The device is portable, disposable, can be used anytime, anywhere discreetly without the need of medical assistance, running water, or other utensils or vesicle containing solutions.

The nasal swab device can be packaged in, held in, delivered in, wrapped in, parceled in, a plastic or paper or metallic (or other synthetic based material) container/packet with tear off or seal access. The packet can contain antiseptic(s) that soak the swab(s) of the device in a various fashions. The package or kit according to the invention can contain one, two, or more of the nasal swab devices each sealed in a separate packet.

In accordance with the provisions of the patent statutes, the invention has been described in what is considered to represent its preferred embodiment. However, it should be noted that the invention can be practiced otherwise than as specifically illustrated and described without departing from its spirit or scope.

1. A 2-step nasal swab kit for cleaning and sanitizing human nose nasal vestibules, the nasal swab kit comprising:
a pair of nasal swab devices, each of the devices having a body from which two spaced apart prongs extend, and each of the prongs having a free end to which a swab is attached;
wherein the swabs of a first of the devices are saturated with at least one cleaning ingredient and the swabs of a second of the devices are saturated with a sanitizing ingredient or antibiotic;
a columellar guard formed as an edge of the body or extending from at least one of the prongs; and
a first packet inside which the first device is sealed and a second packet inside which the second device is sealed.

2. The nasal swab kit according to claim 1 wherein the first packet and the second packet each have a notch formed in an edge thereof providing starting points for tearing open the packets.

3. The nasal swab kit according to claim 1 wherein the first packet and the second packet are conjoined at a perforated seam.

4. The nasal swab kit according to claim 1 wherein the at least one cleaning ingredient is selected from sterile water, glycerin, sodium chloride, sodium benzoate, PEG 60 hydrogenated castor oil, cocamidopropyl betaine, lavender oil, ethyl alcohol, and isopropyl alcohol.

5. The nasal swab kit according to claim 4 wherein the swabs of the first device are saturated with sterile water and ethyl alcohol 62% or isopropyl alcohol 70%.

6. The nasal swab kit according to claim 1 wherein the sanitizing ingredient is mupirocin, a PEG carrier agent, and sterile water or chlorhexidine, an associated carrying agent, and sterile water.

7. The nasal swab kit according to claim 6 wherein the swabs of the second device are saturated with mupirocin in a range of 1-10%, a PEG carrier agent, and sterile water or chlorhexidine, an associated carrying agent, and sterile water.

8. The nasal swab kit according to claim 1 wherein the first packet and the second packet are constructed from a material selected from a paper, a paper product, a plastic, a metal and a synthetic product.

9. The nasal swab kit according to claim 8 wherein the material is biodegradable.

10. The nasal swab kit according to claim 1 wherein the swabs are made of an absorbent material including any of cotton or other natural product, foam, sponge, synthetic fiber and gauze.

11. The nasal swab kit according to claim 1 wherein the first packet is identified by a “step 1 clean” label on an outside surface thereof and the second packet is identified by a “step 2 sanitize” label on an outside surface thereof.

12. A 2-step method for cleaning and sanitizing two nasal vestibules of a human nose, the method comprising the steps of:
simultaneously cleaning a nasal vestibular lining of carriage in the two nasal vestibules using a first nasal swab device saturated with at least one cleaning ingredient; and
then simultaneously sanitizing the cleaned nasal vestibular linings using a second nasal swab device saturated with a sanitizing ingredient.

13. The method according to claim 12 wherein each of the cleaning step and the sanitizing step is performed by inserting swabs of the respective first and second devices into the two nasal vestibules and placing the swabs against the linings, then rotating the double swabs in a circular motion by moving along a circular horizontal path without rotating about a longitudinal axis of the respective device, each of the cleaning step and the sanitizing step being performed for 30 seconds.

14. The method according to claim 12 wherein the first nasal swab device has a pair of swabs saturated with at least one of sterile water, glycerin, sodium chloride, sodium benzoate, PEG 60 hydrogenated castor oil, cocamidopropyl betaine, lavender oil, ethyl alcohol isopropyl alcohol.

15. The method according to claim 12 wherein the second nasal swab device has a pair of swabs saturated with mupirocin in a range of 1-10%, a PEG carrier agent, and sterile water or chlorhexidine, an associated carrying agent, and sterile water.

16. A 2-step nasal swab kit for cleaning and sanitizing human nose nasal vestibules, the nasal swab kit comprising:
a pair of nasal swab devices, each of the devices having a body from which two spaced apart prongs extend, and each of the prongs having a free end to which a swab is attached;
wherein the swabs of a first of the devices are saturated with at least one cleaning ingredient and the swabs of a second of the devices are saturated with a sanitizing ingredient;
a first packet inside which the first device is sealed and a second packet inside which the second device is sealed;
wherein the first packet and the second packet are conjoined at a perforated seam; and
wherein the first packet and the second packet each have a notch formed in an edge thereof providing starting points for tearing open the packets.

17. The nasal swab kit according to claim 16 wherein the at least one cleaning ingredient is selected from sterile water, glycerin, sodium chloride, sodium benzoate, PEG 60 hydrogenated castor oil, cocamidopropyl betaine, lavender oil, ethyl alcohol, isopropyl alcohol and sterile water, and the sanitizing ingredient is mupirocin, a PEG carrier agent, and sterile water or chlorhexidine, an associated carrying agent, and sterile water.

18. The nasal swab kit according to claim 16 wherein the swabs of the first device are saturated with at least one of sterile water, glycerin, sodium chloride, sodium benzoate, PEG 60 hydrogenated castor oil, cocamidopropyl betaine, lavender oil, ethyl alcohol 62%, and isopropyl alcohol 70%.

19. The nasal swab kit according to claim 16 wherein the swabs of the second device are saturated with mupirocin in a range of 1-10%, a PEG carrier agent, and sterile water or chlorhexidine, an associated carrying agent, and sterile water.

20. The nasal swab kit according to claim 16 wherein the first packet is identified by a “step 1 clean” label on an outside surface thereof and the second packet is identified by a “step 2 sanitize” label on an outside surface thereof.
